(19) United States
(12) Patent Application Publication
(10) Pub. No.: US 2016/0081965 A1
(43) Pub. Date: Mar. 24, 2016

(54) ORAL PHARMACEUTICAL COMPOSITION OF ISOTRETINOIN

(71) Applicant: Sun Pharmaceutical Industries Limited, Mumbai (IN)

(72) Inventors: Rathinasabapathy VENKATESHWARAN, Madurai (IN); Sumit MADAN, New Delhi (IN); Harish Kumar MADAN, Sonepat (IN); Ravi KOCHHAR, Gurgaon (IN); Simon Santosh JENA, Ranchi (IN); Rajesh RAO, Deoria (IN); Anuj Kumar FANDA, Ghaziabad (IN); Romi Barat SINGH, Varanasi (IN)

(21) Appl. No.: 14/958,337

(22) Filed: Dec. 3, 2015

Related U.S. Application Data
(63) Continuation of application No. PCT/IB2015/054088, filed on May 29, 2015.

(30) Foreign Application Priority Data
Jun. 2, 2014 (IN) 1456/DEL/2014
Jun. 30, 2014 (IN) 1737/DEL/2014
Dec. 30, 2014 (IN) 4002/DEL/2014

(51) Int. Cl. A61K 31/203; A61K 9/48; A61K 47/02; A61K 47/14; A61K 47/12; A61K 47/32; A61K 47/22; A61K 47/24; A61K 9/00; A61K 47/10

(52) U.S. Cl. CPC A61K 31/203; A61K 9/0053; A61K 9/4833; A61K 47/02; A61K 47/10; A61K 47/12; A61K 47/32; A61K 47/22; A61K 47/24; A61K 47/14

(57) ABSTRACT

The present invention provides an oral pharmaceutical composition of isotretinoin with a reduced dose. The present invention further relates to a process for preparing the oral pharmaceutical composition of the present invention.

### FIELD OF THE INVENTION

The present invention provides an oral pharmaceutical composition of isotretinoin with a reduced dose. The present invention further relates to a process for preparing the oral pharmaceutical composition of the present invention.

### BACKGROUND OF THE INVENTION

Isotretinoin is a retinoid (also known as 13-cis retinoic acid). Owing to its low water solubility, the oral bioavailability of isotretinoin is low. PCT Publication No. WO 00/25772 discloses that the presently marketed formulation of isotretinoin, i.e., Accutane®, contains isotretinoin at a mean particle size of about 100 μm resulting in only 20% oral bioavailability. Therefore, this application discloses a formulation of isotretinoin having a reduced particle size, thereby enhancing the oral bioavailability.

U.S. Pat. Nos. 7,435,427 and 8,367,102 cover the marketed formulation of Absorica®. These patents disclose capsules comprising a semi-solid suspension of isotretinoin containing at least two lipidic excipients, one having an HLB value equal to or greater than 10 and the other being an oily vehicle. These patents are based on the use of the “Lidose technology” to provide a formulation of isotretinoin with enhanced bioavailability.

Isotretinoin has a very high teratogenic potential. This drug may be prescribed only by or under the supervision of a consultant dermatologist. Therefore, reduction of dose in case of such a teratogenic drug is highly beneficial. The present inventors have developed an oral pharmaceutical composition of isotretinoin which has a reduced but effective dose in comparison to the already marketed formulations of isotretinoin, i.e., Roaccutane® and Absorica®/Epuris™.

### SUMMARY OF THE INVENTION

The present invention provides an oral pharmaceutical composition of isotretinoin with a reduced dose. The oral pharmaceutical composition of the present invention comprises isotretinoin and a solvent selected from the group comprising:
i) a monoalkyl ether of diethylene glycol having a general formula C₄H₉O₃(CₙH₂ₙ₊₁), wherein n is 1-4;
ii) an oily vehicle;
iii) optionally ethanol; or
iv) a combination thereof.

The composition is in the form of a solution which is further filled into capsules. The present invention further provides a process for preparing said oral pharmaceutical composition. It also provides a method of treating acne by administering said oral pharmaceutical composition.

### DETAILED DESCRIPTION OF THE INVENTION

In one aspect, the present invention provides an oral pharmaceutical composition comprising isotretinoin and a solvent selected from the group comprising:
i) a monoalkyl ether of diethylene glycol having a general formula C₄H₉O₃(CₙH₂ₙ₊₁), wherein n is 1-4;
ii) an oily vehicle;
iii) optionally ethanol; or
iv) a combination thereof.

In one embodiment of the above aspect, the solvent is present in an amount of about 1% to about 99% by total weight of the composition; preferably in an amount of about 10% w/w to about 90% w/w by total weight of the composition.

In one embodiment of the above aspect, said composition, when administered orally to a patient in need thereof, provides an equivalent efficacy at a lower dose of isotretinoin in comparison to the marketed Epuris™ formulation.

In one embodiment of the above aspect, the dose of isotretinoin is reduced by at least 10% in comparison to the marketed Epuris™ formulation.

In one embodiment of the above aspect, the dose of isotretinoin is reduced by at least 20% in comparison to the marketed Epuris™ formulation.

In one embodiment of the above aspect said composition exhibits improved pharmacokinetic profile as compared to Epuris™ formulation under fed as well as fasting conditions, wherein the pharmacokinetic profile is defined by Cₘₐₓ and AUC.

In another embodiment of the above aspect, said monoalkyl ether of diethylene glycol having a general formula C₄H₉O₃(CₙH₂ₙ₊₁), wherein n is 1-4 includes, but is not limited to, include diethylene glycol monoethyl ether, diethylene glycol monomethyl ether, and mixtures thereof.

In another embodiment of the above aspect, said oily vehicle includes, but is not limited to, fatty acids, fatty acid esters, and vegetable oils.

The fatty acids include, but are not limited to, saturated-, mono-, or di-unsaturated acids, for example, oleic acid, linoleic acid, caprylic acid, caproic acid, and mixtures thereof.

The fatty acid esters include, but are not limited to, polyol esters of medium chain fatty acids selected from esters and mixed esters of glycerol, propylene glycol, polyglycerol, and polyethylene glycol with medium chain fatty acids, phosphatidyl choline with medium chain glycerides, for example caprylic and capric mono-diglyceride esters such as Capmul® MCM, Capmul® MCM C8, glycerol caprylate caprate (Captex® 355), propylene glycol monocaprylate (Capmul® PG-8), ethyl oleate, and mixtures thereof.

The vegetable oils include, but are not limited to, groundnut oil, olive oil, soybean oil, safflower oil, sunflower oil, palm oil, sesame oil, canola oil, corn oil, and mixtures thereof.

In one embodiment of the above aspect, said composition further comprises a surfactant, a co-surfactant or a co-solvent, a hydrophilic polymer, a basic substance, a preservative, and/or an antioxidant.

The surfactants include, but are not limited to, lecithin; sorbitan esters; polysorbates prepared from lauric, palmitic, stearic, and oleic acids; dioctyl sodium sulfosuccinate (DOSS); docusate sodium; sodium lauryl sulfate; Span® 20 and 80; macrogol ethers such as cetomacrogol 1000; polyoxyethylene castor oil derivatives; polyoxyethylene sorbitan fatty acid esters such as Tween®; polyoxyethylene stearates; poloxamers such as Pluronic® F-68 and Pluronic® F.108; macrogolglycerol esters such as Cremophor® EL or Kolliphor® EL; glycerides esters such as lauroyl polyoxyl-32 glycerides (Gelucire®); and mixtures thereof.

The co-surfactants/co-solvents include, but are not limited to, short chain mono-, di-, and polyhydric alcohols, such as ethanol, benzyl alcohol, glycerol, propylene glycol, propylene carbonate, polyethylene glycol with an average molecular weight of about 200 to about 10,000, polyethylene glycol esters such as Labrafil® M1944CS, polyglyceryl-3 dioleate, diethylene glycol monoethyl ether such as Transcutol® HP, and mixtures thereof.

The hydrophilic polymers include, but are not limited to, hydroxypropyl cellulose, hydroxypropylmethyl cellulose, hydroxyethyl cellulose, hydroxymethyl cellulose, carboxymethyl cellulose, methyl cellulose, sodiumcarboxymethyl cellulose, polyvinylpyrrolidone, polysaccharides, gums, alginates, acrylic acid derivatives, and mixtures thereof.

The basic substances include, but are not limited to, inorganic or organic bases, including sodium hydroxide, potassium hydroxide, sodium carbonate or bicarbonate, potassium carbonate or bicarbonate, lithium hydroxide, triethylamine, meglumine, methylamine, and mixtures thereof.

The preservatives include, but are not limited to, methyl paraben, ethyl paraben, propyl paraben, butyl paraben, benzoic acid, sodium benzoate, benzyl alcohol, sorbic acid, potassium sorbate, and mixtures thereof.

The antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof.

In one embodiment of the above aspect, the oral pharmaceutical composition comprises:
(a) isotretinoin;
(b) a basic substance; and
(c) diethylene glycol monoethyl ether.

In another embodiment of the above aspect, the oral pharmaceutical composition comprises:
(a) isotretinoin;
(b) a basic substance; and
(c) a combination of ethanol and an oily vehicle.

In one embodiment of the above aspect, said composition comprises isotretinoin in an amount of about 1 mg to 100 mg, 5 mg to 50 mg, 10 mg to 40 mg, 9 mg to 36 mg, or 8 mg to 32 mg.

In another embodiment of the above aspect, said composition comprises isotretinoin in an amount of about 40 mg.

In another embodiment of the above aspect, said composition comprises isotretinoin in an amount of about 36 mg.

In another embodiment of the above aspect, said composition comprises isotretinoin in an amount of about 32 mg.

In another embodiment of the above aspect, said composition comprises isotretinoin in an amount of about 16 mg.

In yet another embodiment of the above aspect, said composition is in the form of a solution which is further filled into capsules.

In yet another embodiment of the above aspect, said composition is in the form of a self nano-emulsifying drug delivery system (SNEDDS) or self micro-emulsifying drug delivery system (SMEDDS).

In yet another embodiment of the above aspect, said composition is in the form of a self nano-emulsifying drug delivery system (SNEDDS) comprising:
(a) isotretinoin;
(b) a surfactant;
(c) a co-surfactant or a co-solvent; and
(d) an oily phase.

In yet another embodiment of the above aspect, said SNEDDS is a nano-emulsion with globule size less than 1 μm, preferably less than 200 nm, more preferably less than 100 nm.

The ratio of isotretinoin to the oily phase in the said SNEDDS ranges from about 0.04 to about 0.35.

The amount of oily phase in the said SNEDDS ranges from about 10% w/w to about 25% w/w by total weight of the composition.

The amount of surfactant in the said SNEDDS ranges from about 5% w/w to about 55% w/w by total weight of the composition.

The amount of co-surfactant or co-solvent in the said SNEDDS ranges from about 15% w/w to about 75% w/w by total weight of the composition.

In yet another embodiment of the above aspect, said oral pharmaceutical composition is stable when stored at 40° C. and 75% relative humidity or at 25° C. and 60% relative humidity for a period of at least three months or to the extent necessary for the use of the composition.

In another aspect, the present invention provides a process for preparing an oral pharmaceutical composition comprising isotretinoin, wherein said process comprises:
a) dissolving one of more excipients in the solvent selected from the group comprising:
   i) a monoalkyl ether of diethylene glycol having a general formula C₄H₉O₃(CₙH₂ₙ₊₁), wherein n is 1-4;
   ii) an oily vehicle;
   iii) optionally ethanol; or
   iv) a combination thereof;
(b) dissolving isotretinoin in the solution of step (a) to form a clear solution;
(c) filling the solution of step (b) into capsules.

In still another aspect, the present invention provides a method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging, by administering to the individual in need thereof the oral pharmaceutical composition of the present invention.

In one embodiment of the above aspect, the present invention provides a method of treating acne by administering to the individual in need thereof the oral pharmaceutical composition of the present invention.

The term “isotretinoin” refers to isotretinoin in its crystalline or amorphous form, its esters, salts, or derivatives thereof.

The bioequivalence is established by comparing pharmacokinetic parameters for example, AUC and Cₘₐₓ of the pharmaceutical composition of the present invention with Epuris™ in healthy human subjects in fed as well as fasting conditions.

The term “AUC” refers to the area under the time/plasma concentration curve after administration of the pharmaceutical composition. AUC_{0-infinity} denotes the area under the plasma concentration versus time curve from time 0 to infinity; AUC₀₋ₜ denotes the area under the plasma concentration versus time curve from time 0 to time t.

The term “Cₘₐₓ” refers to the maximum concentration of isotretinoin in the blood following administration of the pharmaceutical composition.

The term “tₘₐₓ” refers to the time in hours when Cₘₐₓ is achieved following administration of the pharmaceutical composition.

The term “food effect” as used herein means food-drug interactions which either decrease or increase the extent of drug absorption. It refers to a relative difference in AUC, Cₘₐₓ, and/or tₘₐₓ of a drug, when said drug or a formulation thereof is administered orally to a human, concomitantly with food or in a fed state as compared to when administered in a fasted state or without food.

In certain embodiments, the pharmaceutical composition of the present invention has a reduced food effect, in that when the composition is administered orally to a human concomitantly with food or in a fed state, it has about the same in AUC, Cₘₐₓ, and/or tₘₐₓ as compared to the same values when the same composition is administered in a fasted state or without food.

The term “stable,” as used herein, refers to chemical stability, wherein not more than 1.5% w/w of total related substances are formed on storage at accelerated conditions of stability at 40° C. and 75% relative humidity or at 25° C. and 60% relative humidity for a period of at least three months or to the extent necessary for use of the composition.

The invention may be further illustrated by the following examples, which are for illustrative purposes only and should not be construed as limiting the scope of the invention in any way.

### EXAMPLES

### Example 1

| S. No. | Ingredients | Quantity (% w/w) |
|---|---|---|
| 1 | Isotretinoin | 4.00 |
| 2 | Sodium hydroxide | 0.57 |
| 3 | Butylated hydroxy anisole | 0.10 |
| 4 | Diethylene glycol monoethyl ether | 95.33 |

### Procedure:

1. Sodium hydroxide was dissolved in diethylene glycol monoethyl ether.
2. Butylated hydroxy anisole was dissolved in the solution of step 1.
3. Isotretinoin was dissolved in the solution of step 2 to form a clear solution.
4. The solution of step 3 was filled into capsules.

### Example 2

### Part I

| S. No. | Ingredients | Quantity (% w/w) |
|---|---|---|
| 1 | Isotretinoin | 1.75 |
| 2 | Oleic acid | 98.03 |
| 3 | Butylated hydroxy anisole | 0.22 |

### Procedure—Part I:

1. Butylated hydroxy anisole was dissolved in oleic acid.
2. Isotretinoin was dissolved in the solution of step 1 to form a clear solution.

### Part II

| S. No. | Ingredients | Quantity (% w/w) |
|---|---|---|
| 1 | Isotretinoin | 8.85 |
| 2 | Ethanol | 88.50 |
| 3 | Sodium hydroxide | 2.65 |

### Procedure—Part II:

1. Sodium hydroxide was dissolved in ethanol.
2. Isotretinoin was dissolved in the solution of step 1 to form a clear solution.

### Procedure—Part III:

1. 61.79% w/v of the solution of Part I and 38.21% w/v of the solution of Part II were mixed together to obtain a clear solution.
2. The solution of step 1 was filled into capsules.

### Example 3

| S. No. | Ingredients | Quantity (% w/w) |
|---|---|---|
| 1 | Isotretinoin | 2.00 |
| 2 | Diethylene glycol monoethyl ether | 45.45 |
| 3 | Butylated hydroxy anisole | 0.36 |
| 4 | Povidone K-90 | 4.54 |
| 5 | Oleic acid | 45.45 |
| 6 | Lauroyl polyoxyl-32 glyceride (Gelucire ® | 2.18 |
| | 44/14) | |

### Procedure:

1. Butylated hydroxy anisole and isotretinoin (⅔ of the total quantity) were dissolved in diethylene glycol monoethyl ether to form a clear solution.
2. Povidone was added to the solution of step 1 while stirring to form a clear solution.
3. Oleic acid was taken in a stainless steel container and heated to between 50° C. and 60° C.
4. Lauroyl polyoxyl-32 glyceride was added while stirring into the oleic acid of step 3 to form a clear solution.
5. Isotretinoin (remaining ⅓ quantity) was added while stirring to the solution of step 4 to form a clear solution.
6. The solutions of step 2 and step 5 were mixed while stirring to form a clear solution.
7. The solution of step 6 was filled into hard gelatin capsules.

### Dissolution Studies

The pharmaceutical composition of Example 3 (Test, 16 mg of isotretinoin) was compared with the marketed formulation of isotretinoin (Reference, 20 mg Epuris™ capsules) for the release profile in FDA recommended dissolution medium as given below:

| | |
|---|---|
| Dissolution Media | pH 7.8 phosphate buffer with 0.5% w/v |
| | N,N-dimethyl dodecylamine N-oxide |
| Apparatus RPM/Vol | USP Type I (20 mesh basket)/100/900 mL |

| | % of Drug Released in time (minutes) | | | | | | | | |
|---|---|---|---|---|---|---|---|---|---|
| Sample | 10 | 20 | 30 | 45 | 60 | 90 | 150 | 180 | 210 |
| Test | 10 | 16 | 24 | 36 | 50 | 64 | 88 | 97 | 102 |
| Reference | 0 | 3 | 10 | 37 | 71 | 93 | 110 | 101 | 101 |

### Pharmacokinetic Study Under Fed Conditions

The pharmaceutical composition of Example 3 (Test, 16 mg of isotretinoin) was compared with the marketed formulation of isotretinoin (Reference; 20 mg Epuris™ capsules) under fed conditions in 12 healthy adult male subjects.

Values for various pharmacokinetic parameters, including observed Cₘₐₓ, AUC₀₋ₜ and AUC_{0-inf}, were calculated and are provided in Table 1 below.

TABLE 1
Comparative Pharmacokinetic Data for Test and Reference in 12 Healthy
Adult Human Male Subjects:

| | In Cₘₐₓ | In AUC₀₋ₜ | In AUC_{0-inf} |
|---|---|---|---|
| Ratio (T/R) | 124.26 | 88.08 | 89.50 |
| 90% CI | 106.98-144.33 | 82.96-93.52 | 84.56-94.73 |

### Pharmacokinetic Study Comparing the Formulation of Example 3 Under Fed and Fasting Conditions

The pharmaceutical composition of Example 3 (16 mg Test capsule) was compared in fed and fasting conditions in 12 healthy adult male subjects.

Values for various pharmacokinetic parameters, including observed Cₘₐₓ, AUC₀₋ₜ, and AUC_{0-inf} were calculated and are provided in Table 2 below.

Test (A): Isotretinoin 16 mg capsules (Example 3) under fasting conditions
Test (B): Isotretinoin 16 mg capsules (Example 3) under fed conditions

TABLE 2
Comparative Pharmacokinetic Data for Test (B) vs Test (A) in 12 Healthy
Adult Human Male Subjects:

| | | |
|---|---|---|
| In Cₘₐₓ | In AUC₀₋ₜ | In AUC_{0-inf} |

| | | | |
|---|---|---|---|
| Ratio (B/A) | 80.62 | 106.02 | 107.62 |
| 90% CI | 70.01-92.82 | 100.21-112.18 | 102.02-113.52 |

### Conclusion

Enhanced bioavailability of test in comparison to reference.
Negligible impact of food on extent of absorption for test prototype
Rate of absorption significantly impacted relative to reference.

### Example 4

| S. No. | Ingredients | Quantity (% w/w) |
|---|---|---|
| 1 | Isotretinoin | 3.19 |
| 2 | Diethylene glycol monoethyl ether | 46.98 |
| 3 | Butylated hydroxy anisole | 0.23 |
| 4 | Stearyl macrogol glyceride | 2.59 |
| 5 | Phosphatidyl choline with medium claim | 46.98 |
| | triglycerides | |

### Procedure:

1. Butylated hydroxy anisole was dissolved in diethylene glycol monoethyl ether to form a clear solution.
2. The solution of step 1 was heated to a temperature of between 50° C. and 60° C.
3. Stearyl macrogol glyceride was added to the solution of step 2 while stirring to form a clear solution.
4. The solution of step 3 was cooled to room temperature.
5. Phosphodityl choline with medium chain triglycerides was added to the solution of step 4 while stirring to form a clear solution.
6. Isotretinoin was added to the solution of step 5 while stirring to form a clear solution.
7. The solution of step 6 was filled into hard gelatin capsules.

### Example 5

| S. No. | Ingredients | Quantity (% w/w) |
|---|---|---|
| 1 | Isotretinoin | 3.00 |
| 2 | Propylene glycol monocaprylate | 21.25 |
| 3 | Diethylene glycol monoethyl ether | 25.00 |
| 4 | Macrogolglycerol ricinoleate | 50.62 |
| 5 | Butylated hydroxyl toluene | 0.08 |
| 6 | Propyl gallate | 0.05 |

### Procedure:

1. Propylene glycol monocaprylate, diethylene glycol monoethyl ether, and macrogolglycerol ricinoleate were mixed with stirring to form a solution.
2. Butylated hydroxyl toluene and propyl gallate were dissolved into the solution of step 1 while stirring.
3. Isotretinoin was dissolved into the solution of step 2 while stirring.
4. The solution of step 3 was filled into capsules.

### Example 6

| S. No. | Ingredients | Quantity (% w/w) |
|---|---|---|
| 1 | Isotretinoin | 3.00 |
| 2 | Glyceryl caprylate/caprate | 21.25 |
| 3 | Diethylene glycol monoethyl ether | 25.00 |
| 4 | Macrogolglycerol ricinoleate | 50.62 |
| 5 | Butylated hydroxyl toluene | 0.08 |
| 6 | Propyl gallate | 0.05 |

### Procedure:

1. Glyceryl caprylate/caprate, diethylene glycol monoethyl ether, and macrogolglycerol ricinoleate were mixed with stirring to form a solution.
2. Butylated hydroxyl toluene and propyl gallate were dissolved into the solution of step 1 while stirring.
3. Isotretinoin was dissolved into the solution of step 2 while stirring.
4. The solution of step 3 was filled into capsules.

### Examples 7-10

| | | Quantity (% w/w) | | | |
|---|---|---|---|---|---|
| S. | | | | | Example |
| No. | Ingredients | Example 7 | Example 8 | Example 9 | 10 |
| 1 | Isotretinoin | 4.00 | 4.00 | 4.00 | 4.00 |
| 2 | Propylene glycol | 21.25 | 18.50 | 17.00 | 14.94 |
| | monocaprylate | | | | |
| 3 | Diethylene glycol | 64.00 | 56.00 | 68.25 | 59.65 |
| | monoethyl ether | | | | |
| 4 | Macrogolglycerol | 10.62 | 21.37 | 10.62 | 21.29 |
| | ricinoleate | | | | |
| 5 | Butylated | 0.08 | 0.08 | 0.08 | 0.07 |
| | hydroxyl toluene | | | | |
| 6 | Propyl gallate | 0.05 | 0.05 | 0.05 | 0.05 |

### Procedure:

1. Propylene glycol monocaprylate, diethylene glycol monoethyl ether, and macrogolglycerol ricinoleate were mixed with stirring to form a solution.
2. Butylated hydroxyl toluene and propyl gallate were dissolved into the solution of step 1 while stirring.
3. Isotretinoin was dissolved into the solution of step 2 while stirring.
4. The solution of step 3 was filled into capsules.

### Examples 11-14

| | | Quantity (% w/w) | | | |
|---|---|---|---|---|---|
| S. | | Example | Example | Example | Example |
| No. | Ingredients | 11 | 12 | 13 | 14 |
| 1 | Isotretinoin | 4.00 | 4.00 | 4.00 | 4.00 |
| 2 | Glyceryl | 21.25 | 18.50 | 17.00 | 14.94 |
| | caprylate/caprate | | | | |
| 3 | Diethylene glycol | 64.00 | 56.00 | 68.25 | 59.65 |
| | monoethyl ether | | | | |
| 4 | Macrogolglycerol | 10.62 | 21.37 | 10.62 | 21.29 |
| | ricinoleate | | | | |
| 5 | Butylated | 0.08 | 0.08 | 0.08 | 0.07 |
| | hydroxyl toluene | | | | |
| 6 | Propyl gallate | 0.05 | 0.05 | 0.05 | 0.05 |

### Procedure:

1. Glyceryl caprylate/caprate, diethylene glycol monoethyl ether, and macrogolglycerol ricinoleate were mixed with stirring to form a solution.
2. Butylated hydroxyl toluene and propyl gallate were dissolved into the solution of step 1 while stirring.
3. Isotretinoin was dissolved into the solution of step 2 while stirring.
4. The solution of step 3 was filled into capsules.

### Examples 15-18

| | | Quantity (% w/w) | | | |
|---|---|---|---|---|---|
| S. | | Example | Example | Example | Example |
| No. | Ingredients | 15 | 16 | 17 | 18 |
| 1 | Isotretinoin | 3.03 | 3.03 | 3.03 | 3.01 |
| 2 | Oleic acid | 21.46 | 18.68 | 17.17 | 15.09 |
| 3 | Diethylene glycol | 64.64 | 56.57 | 68.93 | 60.26 |
| | monoethyl ether | | | | |
| 4 | Kolliphor ® EL | 10.74 | 21.59 | 10.74 | 21.51 |
| 5 | Macrogolglycerol | 0.08 | 0.08 | 0.08 | 0.08 |
| | ricinoleate | | | | |
| 6 | Propyl gallate | 0.05 | 0.05 | 0.05 | 0.05 |

### Procedure:

1. Oleic acid, diethylene glycol monoethyl ether, and macrogolglycerol ricinoleate were mixed with stirring to form a solution.
2. Butylated hydroxyl toluene and propyl gallate were dissolved into the solution of step 1 while stirring.
3. Isotretinoin was dissolved into the solution of step 2.
4. The solution of step 3 was filled into capsules.

1. An oral pharmaceutical composition comprising isotretinoin and a solvent selected from the group consisting of:
i) a monoalkyl ether of diethylene glycol having a general formula C₄H₉O₃(CₙH₂ₙ₊₁), wherein n is 1-4;
ii) an oily vehicle;
iii) optionally ethanol; or
iv) a combination thereof.

2. The oral pharmaceutical composition according to claim 1, wherein said composition, when administered orally to a patient in need thereof, provides an equivalent efficacy at a lower dose of isotretinoin in comparison to the marketed Epuris™ formulation.

3. The oral pharmaceutical composition according to claim 2, wherein the dose of isotretinoin is reduced by at least 10% in comparison to the marketed Epuris™ formulation.

4. The oral pharmaceutical composition according to claim 2, wherein the dose of isotretinoin is reduced by at least 20% in comparison to the marketed Epuris™ formulation.

5. The oral pharmaceutical composition according to claim 1, wherein said composition exhibits improved pharmacokinetic profile as compared to Epuris™ capsules under fed as well as fasting condition, wherein the pharmacokinetic profile is defined by Cₘₐₓ and AUC.

6. The oral pharmaceutical composition according to claim 1, wherein the monoalkyl ether of diethylene glycol has a general formula C₄H₉O₃(CₙH₂ₙ₊₁), wherein n is 1-4, and is selected from the group consisting of di ethylene glycol monoethyl ether, diethylene glycol monomethyl ether, and mixtures thereof.

7. The oral pharmaceutical composition according to claim 1, wherein the oily vehicle is selected from the group consisting of fatty acids, fatty acid esters, or vegetable oils.

8. (canceled)

9. (canceled)

10. (canceled)

11. The oral pharmaceutical composition according to claim 1, wherein the solvent is present in an amount of about 1% w/w to about 99% w/w by total weight of the composition.

12. The oral pharmaceutical composition according to claim 11, wherein the solvent is present in an amount of about 10% w/w to about 90% w/w by total weight of the composition.

13. The oral pharmaceutical composition according to claim 1, wherein said composition further comprises one or more of a surfactant, a co-surfactant or a co-solvent, hydrophilic polymer, a basic substance, a preservative, or an antioxidant.

14. The oral pharmaceutical composition according to claim 13, wherein the surfactant is selected from the group consisting of lecithin; sorbitan esters; polysorbates prepared from lauric, palmitic, stearic, and oleic acid; dioctyl sodium sulfosuccinate (DOSS); docusate sodium; sodium lauryl sulfate; Span® 20 and 80; macrogol ethers; polyoxyethylene sorbitan fatty acid esters; poloxamer; macrogolglycerol esters; and mixtures thereof.

15. The oral pharmaceutical composition according to claim 13, wherein the co-surfactant/co-solvent is selected from the group consisting of short chain mono-, di-, and polyhydric alcohols; polyethylene glycol esters; polyglyceryl-3 dioleate; diethylene glycol monoethyl ether; and mixtures thereof.

16. The oral pharmaceutical composition according to claim 13, wherein the hydrophilic polymer is selected from the group consisting of hydroxy propyl cellulose, hydroxypropylmethyl cellulose, hydroxyethyl cellulose, hydroxymethyl cellulose, carboxymethyl cellulose, methyl cellulose, sodiumcarboxymethyl cellulose, polyvinylpyrrolidone, polysaccharides, gums, alginates, acrylic acid derivatives, and mixtures thereof.

17. The oral pharmaceutical composition according to claim 13, wherein the basic substance is selected from the group consisting of inorganic or organic bases, including sodium hydroxide, potassium hydroxide, sodium carbonate or bicarbonate, potassium carbonate or bicarbonate, lithium hydroxide, triethylamine, meglumine, methylamine, and mixtures thereof.

18. (canceled)

19. (canceled)

20. The oral pharmaceutical composition according to claim 13, wherein said composition comprises:
(a) isotretinoin;
(b) a basic substance; and
(c) diethylene glycol monoethyl ether.

21. The oral pharmaceutical composition according to claim 13, wherein said composition comprises:
(a) isotretinoin;
(b) a basic substance; and
(c) a combination of ethanol and an oily vehicle.

22. The oral pharmaceutical composition according to claim 1, wherein said composition comprises isotretinoin in an amount of about 1 to 100 mg, 5 to 50 mg, 10 to 40 mg, 9 to 36 mg, or 8 to 32 mg.

23. The oral pharmaceutical composition according to claim 22, wherein said composition comprises isotretinoin in an amount of about 40 mg, 36 mg, 32 mg, 28 mg, 24 mg, 20 mg, 16 mg, or 8 mg.

24. (canceled)

25. (canceled)

26. (canceled)

27. The oral pharmaceutical composition according to claim 1, wherein said composition is in the form of a solution which is further filled into capsules.

28. The oral pharmaceutical composition according to claim 1, wherein said composition is in the form of a self nano-emulsifying drug delivery system (SNEDDS) or self micro-emulsifying drug delivery system (SMEDDS).

29. The oral pharmaceutical composition according to claim 28, wherein said composition comprises:
(a) isotretinoin;
(b) a surfactant;
(c) a co-surfactant or a co-solvent; and
(d) an oily phase.

30. The oral pharmaceutical composition according to claim 29, wherein said composition is a nano-emulsion with a globule size of less than 1 μm.

31. The oral pharmaceutical composition according to claim 29, wherein said composition is a nano-emulsion with a globule size of less than 200 nm.

32. The oral pharmaceutical composition according to claim 29, wherein said composition is a nano-emulsion with a globule size of less than 100 nm.

33. The oral pharmaceutical composition according to claim 29, wherein the ratio of isotretinoin to the oily phase ranges from about 0.04 to about 0.35.

34. The oral pharmaceutical composition according to claim 29, wherein the amount of the oily phase ranges from about 10% w/w to about 25% w/w by total weight of the composition, the amount of surfactant ranges from about 5% w/w to about 55% w/w by total weight of the composition, and the amount of co-surfactant or co-solvent ranges from about 15% w/w to about 75% w/w by total weight of the composition.

35. (canceled)

36. (canceled)

37. The oral pharmaceutical composition according to claim 1, wherein said composition is stable when stored at 40° C. and 75% relative humidity or at 25° C. and 60% relative humidity for a period of at least three months.

38. A process for preparing an oral pharmaceutical composition according to claim 1, wherein said process comprises:
(a) dissolving one of more excipients in the solvent selected from the group consisting of:
i) a monoalkyl ether of diethylene glycol having a general formula C₄H₉O₃(CₙH₂ₙ₊₁), wherein n is 1-4;
ii) an oily vehicle;
iii) optionally ethanol; or
iv) a combination thereof;
(b) dissolving isotretinoin in the solution of step (a) to form a clear solution;
(c) filling the solution of step (b) into capsules.

39. The oral pharmaceutical composition according to claim 1, wherein said composition is used for the treatment of acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, psoriasis, cutaneous lupus erythematosus, acne fulminans, squamous cell carcinoma, or cutaneous photoaging.

40. (canceled)

41. (canceled)

42. (canceled)
